(19) United States
(12) Plant Patent Application Publication
(10) Pub. No.: US 2009/0210981 P1
(43) Pub. Date: Aug. 20, 2009

(54) Blueberry plant named 'Azulema'

(50) ;

(75) Inventors: Antonio Abad Alamo, Almonte (ES); Jose Ulf Hayler Lopez, Almonte (ES); Paul M. Lyrene, Micanopy, FL (US)

Correspondence Address: BUCHANAN, INGERSOLL & ROONEY PC, POST OFFICE BOX 1404, ALEXANDRIA, VA, 22313-1404, US

(73) Assignee: ROYAL BERRIES S.L., Almonte (ES)

(21) Appl. No.: 12/289,127

(22) Filed: Oct. 21, 2008

(30) Foreign Application Priority Data
Feb. 14, 2008 (QZ) 2008/0345

(51) Int. Cl. A01H 5/00

(52) U.S. Cl. PLT/157

(57) ABSTRACT

A new and distinct Blueberry cultivar is provided that is the product of a controlled breeding program followed by selection. The cultivar flowers and forms fruit at mid-season. The attractive high quality firm light blue flattened-round berries exhibit a superb aromatic blueberry flavor. The plant is self-fertile, and displays a generally round to vase-shaped growth habit with attractive evergreen foliage. A low chilling requirement is also exhibited.

### BOTANICAL/COMMERCIAL CLASSIFICATION

Vaccinium corymbosum L./Blueberry Plant

### VARIETAL DENOMINATION

cv. Azulema

### SUMMARY OF THE INVENTION

The new Blueberry cultivar of the present invention was the product of controlled artificial pollination carried out in a greenhouse at Greenwood, Fla., U.S.A., wherein two parents were crossed which previously had been studied in the hope that they would contribute the desired characteristics. The female parent (i.e., the seed parent) was the unreleased ‘FL 90-4’ cultivar (non-patented in the United States). The male parent (i.e., pollen parent) was the unreleased ‘FL 96-32’ cultivar (non-patented in the United States). The parentage of the new cultivar can be summarized as follows:

‘FL 90-4’×‘FL 96-32’.

The seeds resulting from the pollination were shipped to Almonte, Huelva, Spain, where they sown during approximately 2000, small plants were obtained which were physically and biologically different from each other and selective research of the progeny was carried out. Selective study during the spring of 2003 resulted in the identification of a single plant of the new cultivar. This plant initially was designated S03-38-01.

It was found that the new Blueberry plant of the present invention displays the following combination of characteristics:
(a) flowers and forms fruit at mid-season,
(b) displays a generally round to vase-shaped growth habit with attractive evergreen foliage,
(c) is self-fertile,
(d) displays a low chilling requirement, and
(e) forms in abundance attractive firm light blue flattened-round berries that exhibit a superb aromatic blueberry flavor and a good shelf life following harvest.

The new cultivar well meets the needs of the horticultural industry and can be grown to advantage for the commercial production of blueberries. It is recommended that the plant be grown under tunnels in warm areas with well drained soils while using evergreen management procedures.

The new cultivar of the present invention can be distinguished from its ancestors and all other Blueberry cultivars known to its originators. When compared to the Star’ cultivar (U.S. Plant Pat. No. 10,676), the ‘Star’ cultivar commonly displays a taller, more upright growth habit. When compared to the ‘Millenia’ cultivar (U.S. Plant Pat. No. 12,816), the ‘Millenia’ cultivar commonly requires cross-pollination unlike the new cultivar of the present invention. When compared to the ‘O'Neal’ cultivar (non-patented in the United States), the ‘O'Neal’ cultivar lacks an evergreen winter stage and forms darker blue berries and requires a longer chill requirement of approximately 500 hours. The new cultivar is less susceptible to Leaf Rust than the ‘Blue Crisp’ cultivar (U.S. Plant Pat. No. 11,033), and is more resistant to Stem Blight than the ‘Biloxi’ cultivar (non-patented in the United States).

The new cultivar has been asexually reproduced by the rooting of softwood cuttings beginning during the summer of 2003 at Almonte, Huelva, Spain. Such asexual propagation has shown that the characteristics of the new cultivar are firmly fixed and are stably transmitted from one generation to another. Accordingly, the new cultivar asexually reproduces in a true to type manner.

The new cultivar has been named ‘Azulema’.

### BRIEF DESCRIPTION OF THE PHOTOGRAPHS

The accompanying photographs show in color as nearly true as it is reasonably possible to make the same in color illustrations of this character, typical plants and plant parts of the new cultivar. The plants which had been asexually reproduced by the rooting of softwood cuttings, and were being grown outdoors at Almonte, Huelva, Spain.

FIG. 1 shows an overall view of a typical flowering plant of the new cultivar where the generally round to vase-shaped growth habit is illustrated.

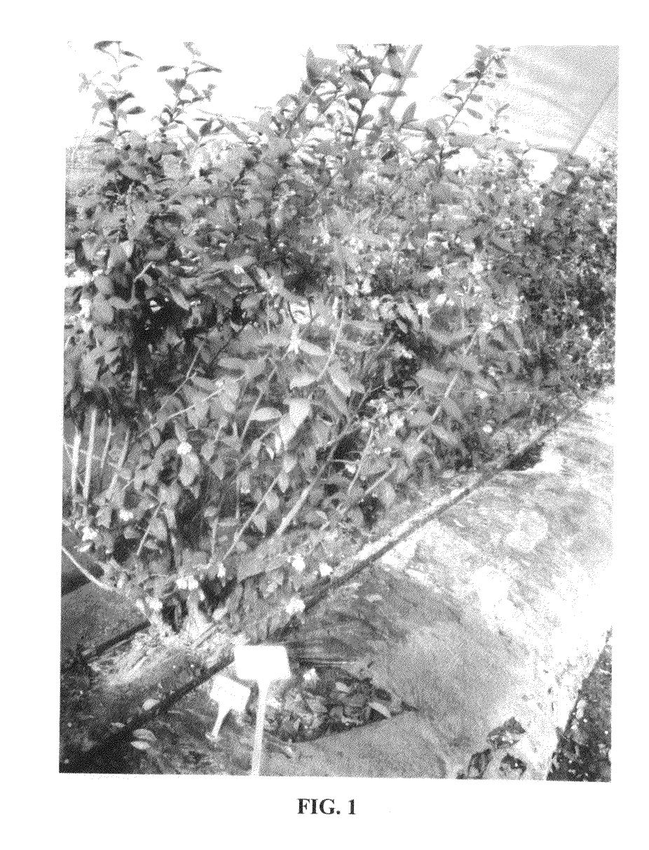

FIG. 2 shows a close view of typical flowers of the new cultivar.

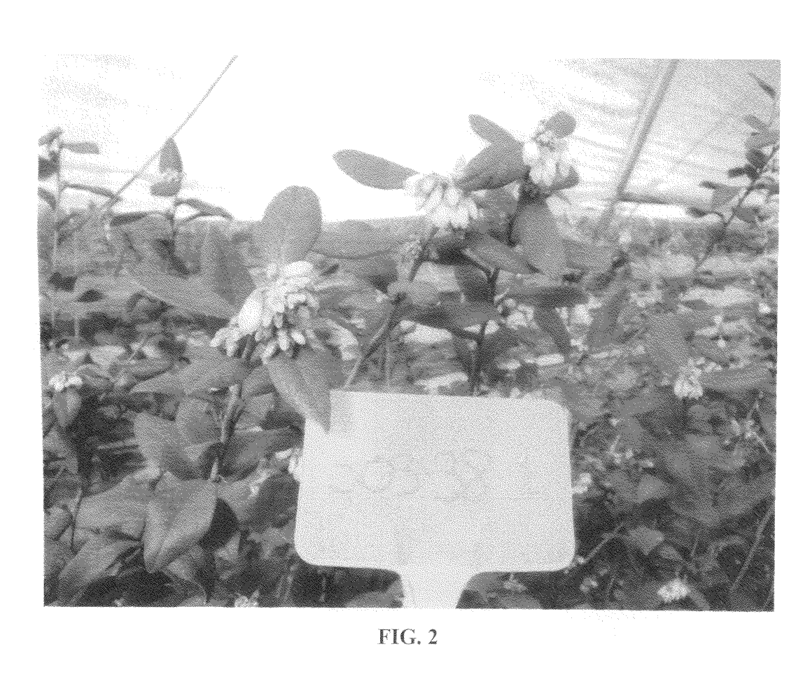

FIG. 3 shows a cluster of typical berries in various stages of development and the foliage of the new variety.

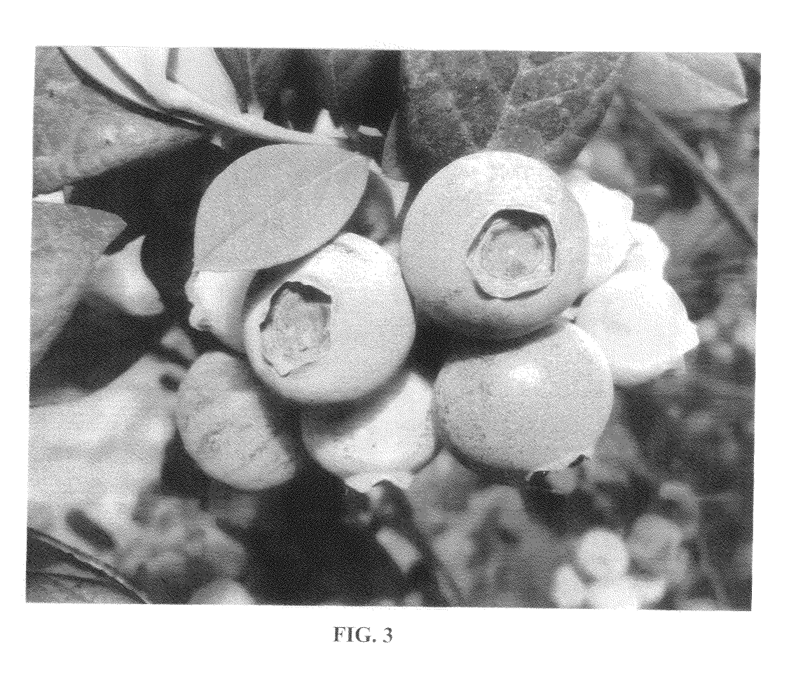

FIG. 4 shows upper (adaxial) surfaces of typical leaves of the new cultivar.

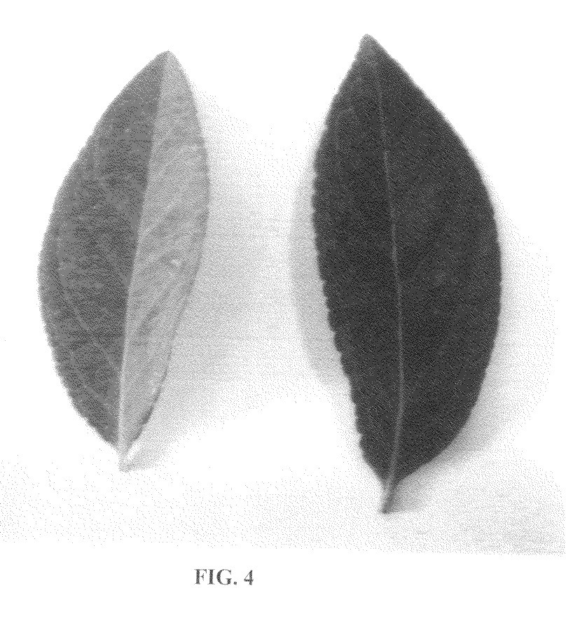

FIG. 5 shows under (abaxial) surfaces of typical leaves of the new cultivar.

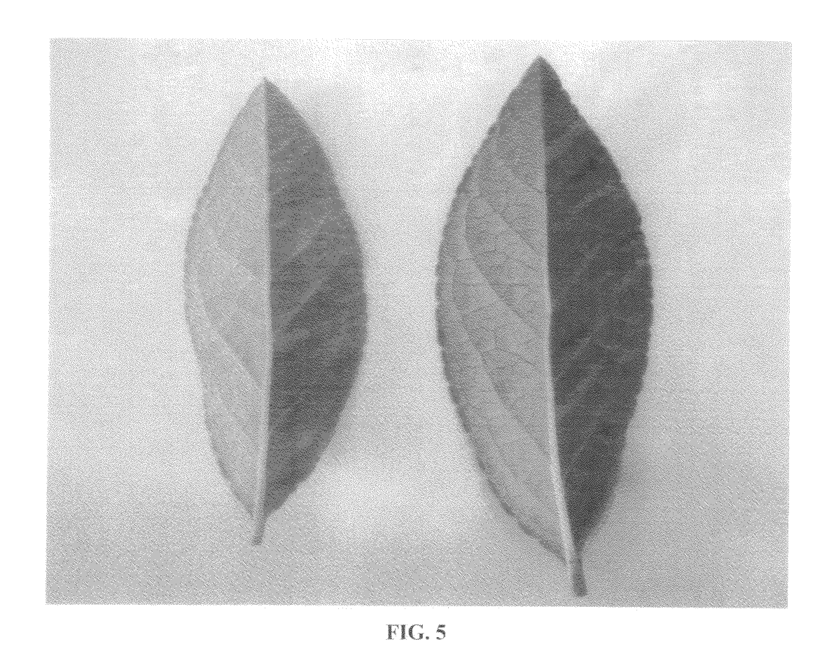

FIG. 6 shows a close view of an array of typical mature berries of the new cultivar.

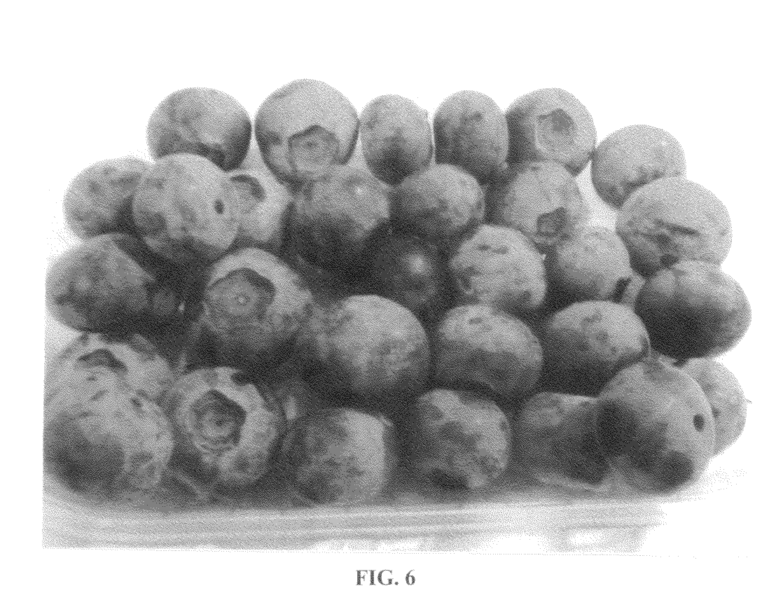

### DETAILED DESCRIPTION

The chart used in the identification of the colors described herein is the R.H.S. Colour Chart of The Royal Horticultural Society, London, England. Ordinary color terms are to be accorded their customary dictionary significance. The description is based on the observation of approximately four-year-old plants of the new cultivar which had been asexually reproduced by the rooting of softwood cuttings while growing outdoors at Almonte, Huelva, Spain.

### Plant:

Growth habit.—generally round to vase-shaped.
Height.—approximately 1.4 m at 4 years of age.
Width.—approximately 3.3 m at 4 years of age.
Foliage retention.—evergreen.
Chill requirement.—commonly less than approximately 300 hours.
Foliage:
   Shape.—narrowly elliptic (as illustrated).
   Length.—commonly approximately 67 mm on average.
   Width.—commonly approximately 33 mm on average.
   Apex.—acute.
   Base.—acute.
   Margin.—crenate.
   Texture.—glabrous and non-glandular.
   Color.—Green Group 137B on the upper (adaxial) surface, and Green Group 138C on the under (abaxial) surface.
Flowers:
   Time.—mid-season, at Almonte, Huelva, Spain, with first flower commonly at approximately December 20^{th}, and 50 percent bloom at approximately February 20^{th}.
   Number.—commonly approximately 7 flowers per bud on average.
   Petals.—5 in number and fused into a corolla tube.
   Fertility.—self-fertile.
   Fragrance.—none.
Fruit:
   Time.—commonly from approximately April 7^{th} to June 15^{th} at Almonte, Huelva, Spain (i.e., approximately 8 weeks).
   Shape.—generally flattened-round.
   Height.—commonly approximately 15 mm on average.
   Width.—commonly approximately 18 mm on average.
   Weight.—approximately 2.65 g/berry on average when plants were 4 years of age.
   Fruit scar.—approximately 1.2 mm in size, dry, and relatively deep (as illustrated).
   Seed number.—commonly approximately 20 per berry on average.
   Seed size.—commonly approximately 1.4 mm in length and approximately 0.9 mm in width on average.
   Immature color.—commonly near Green Group 142D with bloom, and Yellow-Green Group 145A without bloom.
   Mature color.—light blue, Violet Blue Group 97B to 97C with bloom, and Black Group 202A without bloom.
   Productivity.—abundant, approximately 3.73 Kg/plant on average when plants are 4 years of age.
   Flavor.—displays a superb aromatic blueberry flavor.
Development:
   Ability to store.—the fruit stores well under refrigeration, when stored at 8° C. approximately 96 percent of the berries are of good quality 7 days after harvest, and when stored at 20° C. approximately 94 percent of the berries are of good quality 7 days after harvest.
   Disease tolerance.—no special sensitivity to common Blueberry diseases. such as Leaf Rust (Pucciniastrum vaccinii) and Botrytis (Botrytis cinerea) has been encountered during observations to date at Almonte, Huelva, Spain. During observations to date the new cultivar is less susceptible to Leaf Rust than the ‘Blue Crisp’ cultivar, and is more resistant to Stem Blight than the ‘Biloxi’ cultivar.
   Insects.—is susceptible to aphids and thrips.
   Cultural conditions.—is well suited for evergreen management under tunnels.

Plants of the ‘Azulema’ cultivar have not been observed under all possible environmental conditions to date, Accordingly, it is possible that the phenotypic expression may vary somewhat with changes in light intensity and duration, cultural practices, and other environmental conditions without variance in the genotype.

We claim:

1. A new and distinct Blueberry plant that possesses the following combination of characteristics:
(a) flowers and forms fruit at mid-season,
(b) displays a generally round to vase-shaped growth habit with attractive evergreen foliage,
(c) is self-fertile,
(d) displays a low chilling requirement, and
(e) forms in abundance attractive firm light blue flattened-round berries that exhibit a superb aromatic blueberry flavor and a good shelf life following harvest;
substantially as herein shown and described.
